(19) United States
(12) Patent Application Publication
(10) Pub. No.: US 2010/0172839 A1
(43) Pub. Date: Jul. 8, 2010

(54) METHOD AND SYSTEM FOR EVALUATING GASTROINTESTINAL MOTILITY

(75) Inventor: Dwight Sherod Walker, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION

(21) Appl. No.: 12/514,472

(22) PCT Filed: Nov. 12, 2007
(86) PCT No.: PCT/US2007/084378

§ 371 Date: May 12, 2009

Related U.S. Application Data
(60) Provisional application No. 60/866,505, filed on Nov. 20, 2006.

(51) Int. Cl. A61K 49/00; A61B 7/00

(52) U.S. Cl. 424/9.1; 600/586

(57) ABSTRACT

A system and method for evaluating gastrointestinal motility that can be effectively employed to acquire one or more signals associated with acoustic energy (i.e. sound) emanating from an abdominal region of a body and determine at least one gastrointestinal parameter based on the acoustic energy signal(s) is described. The gastrointestinal parameter can include a gastrointestinal event, including gastrointestinal mixing, emptying, contraction and propulsion, and gastrointestinal transit time.

### FIELD OF THE INVENTION

The present invention relates generally to methods for non-invasive assessment of gastrointestinal function.

### BACKGROUND OF THE INVENTION

Advances in the pharmaceutical industry in the past decades have been essential in extending the length and quality of the human life. In addition to new compounds, advances in oral medication formulation and delivery methods have helped improve efficacy while minimizing dosage and reducing side effects. However, the human body's digestive tract is heterogeneous, and differences in digestive enzymes, absorption rates, micro flora, and other factors make certain sites in the gastro-intestinal tract more or less ideal for the delivery of specific medications.

Drug companies have focused considerable efforts in targeted drug delivery, i.e. location and rate of drug delivery within the gastrointestinal (“GI”) tract. These efforts have resulted in variations in the forms of basic delivery designs, e.g., gel capsule vs. hard tablet, coating formulations, etc., and more recently, advanced control over micro- and nana-particle size. While these advances have proven beneficial, the human element remains: the GI system is intensely variable, both inter-and intra-subject. A key variable, gastrointestinal motility and, hence, gastrointestinal (or digestive) transit time, complicates determining the ideal targeted drug delivery.

Gastrointestinal motility also can, and in many instances will, have a significant impact on the clinical evaluation of the efficacy of a pharmaceutical formulation. Indeed, as is well known in the art, if an orally delivered pharmaceutical formulation, e.g., gel capsule containing a pharmaceutical formulation, exits the gastrointestinal tract prior to optimum dissolution and, hence, absorption, the efficacy of the formulation will be greatly diminished. Moreover, it has been found that in some instances, the capsule can remain in the upper gastrointestinal tract (i.e. upper fundus) for extended periods of time (e.g., >5 hrs).

Various methods and systems have been employed to assess gastrointestinal motility and transit time. A commonly employed method comprises gamma scintigraphy. There are, however, several significant drawbacks associated with gamma scintigraphy. One drawback is that the method is presently limited to a small number of facilities and experts due to the issues (and controls) associated with handling radiological substances and the equipment expense. A further drawback is that large scale clinical drug trials are impractical.

Further methods and systems for assessing gastrointestinal motility include the acquisition and evaluation of gastrointestinal sounds. For example, in U.S. Pat. No. 5,301,679, a method and system are disclosed for providing diagnostic information for various diseases, including diseases of the gastrointestinal tract, by capturing body sounds with a microphone placed on the body surface or inserted orally or rectally into the gastrointestinal tract.

Other systems also employ microphones sensitive to gastrointestinal sounds within specific frequency ranges and are exemplified by Dalle, et al., “Computer Analysis In Bowel Sounds”, Computers in Biology and Medicine, Vol. 4 (3-4), pp. 247-254 (Feb. 1975); Sugrue et al., “Computerized Phonoenterography: The Clinical Investigation of the New System”, Journal of Clinical Gastroenterology, Vol. 18, No. 2, pp. 139-144 (1994); Poynard, et al., “Qu'attendre des systemes experts pour le diagnostic des troubles fonctionnes intestinaux”, Gastroenterology Clinical Biology, pp. 45c-48c (1990).

A significant drawback associated with the conventional acoustic methods and systems is that the scope of information that can be derived from the recorded sounds is limited. Indeed, there is little, if any, disclosure directed to the relationship between gastrointestinal sounds and gastrointestinal transit times.

It would therefore be desirable to provide a method and system for evaluating gastrointestinal motility and determining gastrointestinal transit time by abdominal auscultation.

### SUMMARY OF THE INVENTION

Embodiments of the present invention provide systems and methods for evaluating gastrointestinal motility. In some embodiments, the systems and methods can provide a variety of information, including gastrointestinal transit time and other physiological parameters.

In accordance with one aspect of the invention, there is provided a system and method for evaluating gastrointestinal motility that can be effectively employed to acquire one or more signals associated with acoustic energy (i.e. sound) emanating from an abdominal region of a body and determine at least one gastrointestinal parameter based on the acoustic energy signal(s).

In accordance with one embodiment of the invention, there is thus provided a system for monitoring gastrointestinal motility of a subject, comprising: (a) at least one sensor mountable on or in a body region of the subject, the sensor being adapted to sense acoustic energy and generate at least one acoustic energy signal representing the acoustic energy, and (b) a processing unit adapted to receive the acoustic energy signal, the processing unit being further adapted to process the acoustic energy signal and determine the occurrence of a gastrointestinal event.

In one embodiment, the sensor generates a plurality of acoustic energy signals representing the acoustic energy and the processing unit is adapted to receive and process the acoustic energy signals to determine the occurrence of a gastrointestinal event.

In one embodiment of the present invention, the acoustic energy comprises gastrointestinal sounds.

In accordance with another embodiment, there is provided a method of monitoring gastrointestinal motility of a subject, comprising the steps of: (a) sensing acoustic energy generated by the subject's gastrointestinal system, and (b) processing the acoustic energy in order to determine a gastrointestinal parameter.

In one embodiment, the gastrointestinal parameter comprises gastrointestinal transit time.

In accordance with a further embodiment of the invention, there is provided a method for evaluating clinical data derived from a subject, comprising the step of comparing a gastrointestinal parameter of the subject to at least one physiological parameter induced in the subject by the administration of the pharmaceutical to the subject.

In accordance with another embodiment of the invention, there is provided a method for evaluating clinical data derived from a subject, comprising the steps of: (i) orally administering a pharmaceutical to the subject, (ii) monitoring acoustic energy generated by the subject's gastrointestinal system, (iii) generating at least one acoustic energy signal representing the acoustic energy, (iv) processing the acoustic energy signal to determine a gastrointestinal parameter related thereto, and (v) comparing the gastrointestinal parameter to at least one physiological parameter induced in the subject by the administration of the pharmaceutical to the subject.

In one embodiment of the invention, the gastrointestinal parameter comprises gastrointestinal transit time.

In one embodiment, the physiological parameter comprises a pharmacokinetic (PK) characteristic.

In accordance with another embodiment of the invention, there is provided a method for evaluating gastrointestinal motility of a subject, comprising the steps of: (i) orally administering an ingestible to the subject, (ii) monitoring acoustic energy generated by the subject's gastrointestinal system, (iii) generating at least one acoustic energy signal representing the acoustic energy, and (iv) processing the acoustic energy signal to derive a gastrointestinal parameter related thereto.

In one embodiment of the invention, the gastrointestinal parameter comprises gastrointestinal transit time.

### BRIEF DESCRIPTION OF THE DRAWINGS

FIG. 1A is an illustration of a portion of a human torso showing a typical gastrointestinal tract;

FIG. 1B is an illustration of a human stomach;

FIG. 2 is a schematic illustration of a gastrointestinal motility analysis system, according to one embodiment of the invention;

FIG. 3 is a further illustration of the partial human torso shown in FIG. 1, showing the placement of gastrointestinal sound (or acoustic) sensors, according to one embodiment of the invention;

FIG. 4 is a schematic illustration of an analyzer, showing the sub-systems or modules thereof, according to one embodiment of the invention;

FIG. 5 is a further illustration of a portion of a human torso having a system vest disposed thereon, according to one embodiment of the invention;

FIG. 6 is a schematic illustration of a gastrointestinal motility analysis system having additional physiological sensors, according to another embodiment of the invention;

FIG. 7 is a summary of gama scintigraphy results acquired during a gastrointestinal motility study; and

FIGS. 8-14 are graphical illustrations of gastrointestinal sound signals, reflecting gastrointestinal sounds acquired during the gastrointestinal motility study summarized in FIG. 7.

### DETAILED DESCRIPTION OF THE INVENTION

Before describing the present invention in detail, it is to be understood that this invention is not limited to particularly exemplified structures, apparatus, systems, materials or methods as such may, of course, vary. Thus, although a number of apparatus, systems and methods similar or equivalent to those described herein can be used in the practice of the present invention, embodiments of the apparatus, systems and methods according to the present invention are described herein.

It is also to be understood that like referenced characters generally refer to the same parts or elements throughout the views shown in the figures.

Unless defined otherwise, all technical and scientific terms used herein have the same meaning as commonly understood by one having ordinary skill in the art to which the invention pertains. Further, all publications, patents and patent applications cited herein, whether supra or infra, are hereby incorporated by reference in their entirety.

Finally, as used in this specification and the appended claims, the singular forms “a”, “an”, “the” and “one” include plural referents unless the content clearly dictates otherwise. Thus, for example, reference to “a sensor” includes two or more such sensors; reference to “a gastrointestinal event” includes two or more such events and the like.

### Definitions

The term “pharmaceutical composition”, as used herein, is meant to mean and include any compound or composition of matter or combination of constituents, which, when administered to an organism (human or animal) induces a desired pharmacologic and/or physiologic effect. The term therefore encompasses substances traditionally regarded as actives, drugs, prodrugs, and bioactive agents, as well as biopharmaceuticals (e.g., peptides, hormones, nucleic acids, gene constructs, etc.).

The term “pharmaceutical”, as used herein, is meant to mean and include a pharmaceutical composition that precipitates acoustic energy or a gastrointestinal sound (or sounds) from the gastrointestinal tract when orally administered to a human or animal, such as, without limitation, pharmaceutical compositions in the form of hard tablets, gel capsules (hard and soft), caplets and other solid dosage forms.

The term “ingestible”, as used herein, is meant to mean and include any substance or item that precipitates acoustic energy or a gastrointestinal sound (or sounds) from the gastrointestinal tract when orally administered to a human or animal. An “ingestible” can thus comprise a pharmaceutical, as well as a non-pharmaceutical composition, such as, without limitation, a placebo.

The term “gastrointestinal event”, as used herein means and includes an activity or function associated with the gastrointestinal system, including, without limitation, gastrointestinal mixing, emptying, contraction and propulsion. A “gastrointestinal event” can also comprise a mitigating motor complex (MMC) phase.

The term “gastrointestinal parameter”, as used herein, means and includes a characteristic associated with gastrointestinal function, including, without limitation, a gastrointestinal event and gastrointestinal transit time.

The term “gastrointestinal sound”, as used herein, means and includes acoustic energy (and all signals embodied therein) generated by a gastrointestinal event.

The term “gastrointestinal transit time”, as used herein, is meant to mean the motile time through one or more sections of the gastrointestinal tract that can be impacted by the composition of the materials being passed, state of the gastrointestinal tract, psychological stress, gender, and other factors. “Gastrointestinal transit time” is a generic term that can be used to describe the overall gastrointestinal transit time, the fundus-rectal transit time, and various other motile times through one or more sections of the gastrointestinal tract.

The term “overall gastrointestinal transit time”, as used herein, means the motility time of a pharmaceutical or ingestible from the point it is administered via its intended route (e.g., oral, rectal) through the various sections of the gastrointestinal tract and its exit from the body.

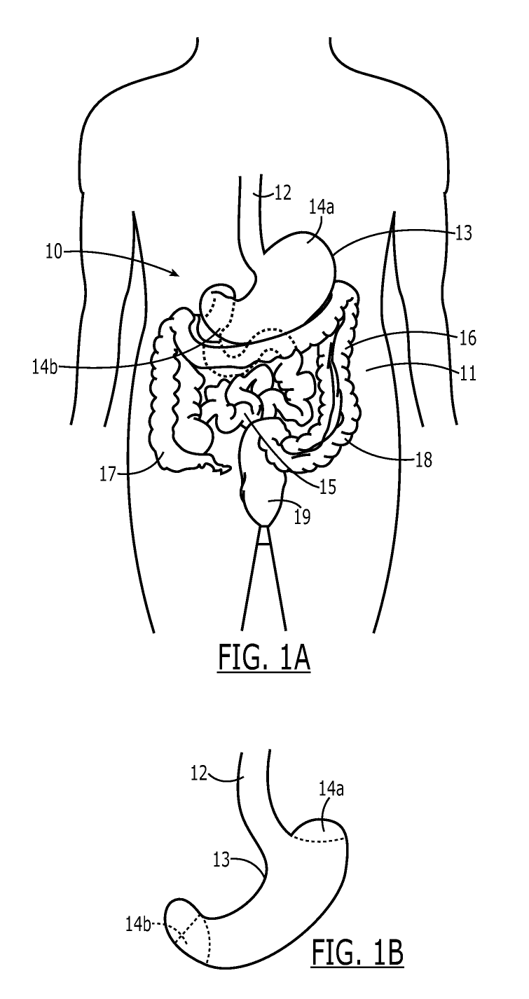

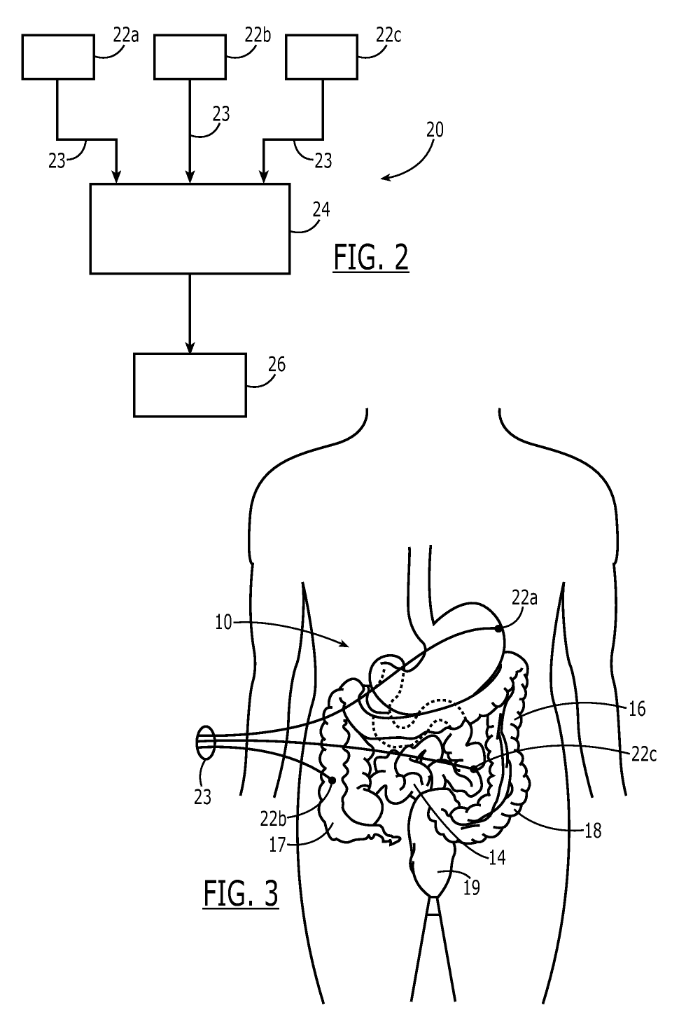

The term “fundus-rectal gastrointestinal transit time”, as used herein, means the motility time of a pharmaceutical or ingestible from entry into the fundus of the stomach through ejection from the rectum (see FIGS. 1A and 1B).

The term “signal voltage envelope”, as used herein, means an envelope that is derived from a plurality of acoustic energy signal voltages. The “signal voltage envelope” has upper and lower boundaries defined by the acoustic energy signal voltages.

The term “signal amplitude envelope”, as used herein, means an envelope that is derived from a plurality of acoustic energy signal amplitudes. The “signal amplitude envelope” has upper and lower boundaries defined by the acoustic energy signal amplitudes.

The term “V_{threshold}”, as used herein, means the minimum voltage at which values may be considered significant. According to the invention, if the signal voltage envelope is below V_{threshold}, there is no response (i.e. the signal is below the detector's sensitivity). If the signal voltage envelope is larger than V_{threshold} for longer than a pre-determined amount of time, the value is deemed significant.

The term “subject”, as used herein, means and includes a human or an animal.

The present invention provides systems and methods for evaluating gastrointestinal motility. As set forth in detail herein, methods and systems of the invention can be effectively employed to acquire one or more signals associated with acoustic energy (i.e. sound) emanating from an abdominal region of a body and determine at least one gastrointestinal parameter based on the acoustic energy signal(s) and/or the onset thereof.

Implementation of the methods and systems of embodiments of the present invention, as described herein, can involve performing or completing selected tasks or steps manually, automatically, or a combination thereof. In some embodiments of the present invention, several selected steps could be implemented by hardware or by software on any operating system or any firmware or a combination thereof. For example, as hardware, selected steps of embodiments of the invention could be implemented as a chip or a circuit. As software, selected steps of embodiments of the invention could be implemented as a plurality of software instructions being executed by a computer using any suitable operating system. In any case, selected steps of the method and system of the invention could be described as being performed by a data processor, such as a computing platform for executing a plurality of instructions.

Referring first to FIG. 1A, there is shown an illustration of a typical gastrointestinal tract (designated generally “10”). As illustrated in FIG. 1, the gastrointestinal tract 10 generally includes the oesophagus or esophagus 12, stomach 13, small intestines 15 and large intestines 16. The large intestines include the cecum 17, colon 18 and rectum 19.

Referring to FIG. 1B, the stomach 13 includes the fundus region (or fundus) 14a and pyloric antrum (or antrum) 14b.

As is well known in the art, gastrointestinal motility (in a normal male/female subject) is typically characterized by the repeated appearance of three distinctive phases, called the migrating motor complex (MMC). Phase 1 comprises a period or phase of no contractions. Phase 2, which follows phase 1, comprises a phase of intermittent, variable-amplitude contractions. Phase 3, which follows phase 2, comprises a phase of repetitive propagating contractions. The mitigating motor complex has an average cycle of 80 to 150 min.

It is also well established and known in the art that distinctive sounds emanate from the gastrointestinal tract during each of the noted phases. See, e.g., T. Tomomasa, et al.,

“Gastrointestinal Sounds and Migrating Motor Complex In Fasted Humans”, The American Journal of Gastroenterology, Vol. 94, No. 2, pp. 374-381 (1999); J. Farrar, et al., “Gastrointestinal Motility as Revealed by Study of Abdominal Sounds”, Gastroenterology, Vol. 29, No. 5, pp. 789-800 (1955); W. B. Cannon, “Auscultation of the Rhythmic Sounds Produced by the Stomach and Intestines”, Laboratory of Physiology, VI, pp. 339-353 (1905).

As indicated above, although there have been various studies relating to gastrointestinal sounds and publications resulting therefrom, there is scant information relating to the relationships between gastrointestinal sounds and the migrating motor complex. There is also very little information relating to the relationship between gastrointestinal sounds and gastrointestinal transit time.

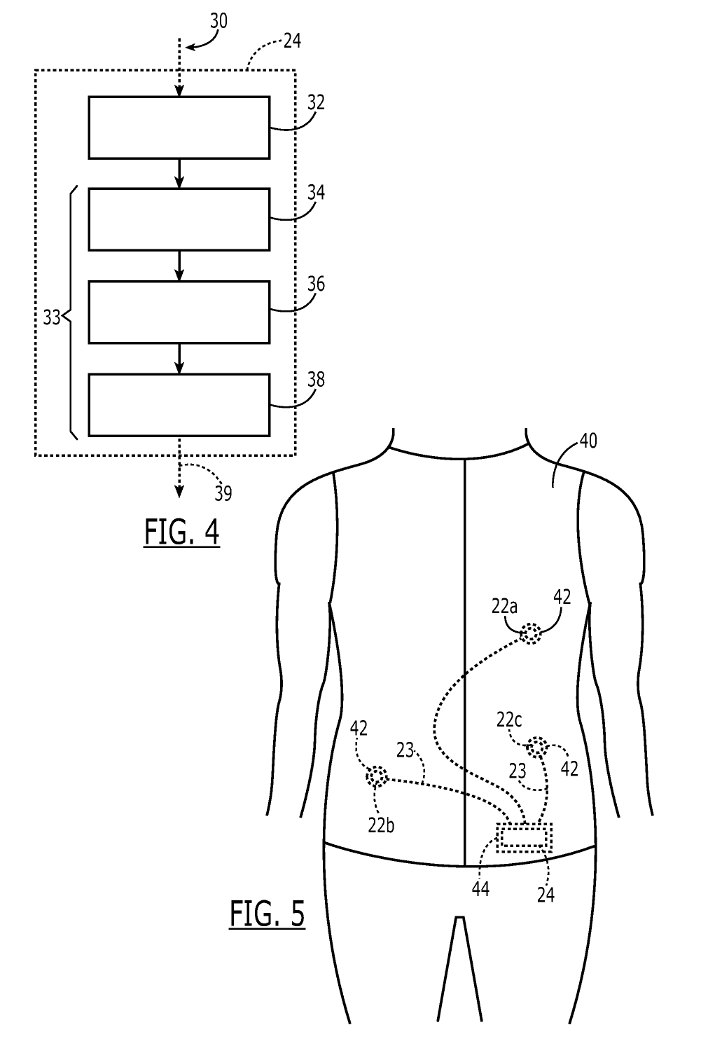

Referring now to FIG. 2, there is shown a schematic illustration of one embodiment of a gastrointestinal motility analysis system 20. As illustrated in FIG. 2, the system 20 includes a plurality of acoustic energy sensors 22a, 22b, 22c and at least one analyzer 24. In the embodiment shown in FIG. 2, the system 20 also includes display means 26.

According to the invention, the sensors 22a, 22b, 22c can independently comprise contact or non-contact transducers that detect vibrations and/or sounds at or near the skin surface and convert these vibrations and/or sounds into electrical signals. Other sensors can include internal sensors, such as intra-esophageal and intra-gastric sensors, that are introduced into the patient using a nasal-gastric tube or the like.

By way of example only, the sensors 22a, 22b, 22c can be electronic stethoscopes, contact microphones, non-contact vibration sensors, such as capacitive or optical sensors, or any other suitable type of sensors. The sensors 22a, 22b, 22c are preferably, but not necessarily, selected to have acoustic impedance that matches the impedance of the skin surface to provide optimal acoustic coupling to the skin surface. Still further, due to background noise and the relatively low amplitude of the vibrations or sounds which are generated at or near the skin surface by gastric sounds, the sensors 22a, 22b, 22c are also preferably, but not necessarily, selected to provide a high signal-to-noise ratio, high sensitivity and/or good ambient noise shrouding capability.

According to the invention, the sensors 22a, 22b, 22c send low level (i.e. low power) electrical signals via wires 23, or any other suitable media, such as wireless radio frequency, infrared, etc., to the analyzer 24.

A suitable sensor that can be employed within the scope of the invention is disclosed in U.S. Pat. No. 6,512,830.

While three sensors are shown in FIG. 2, additional or fewer sensors can be used to detect gastric sounds at multiple locations on the patient's abdomen 11, or any other locations on the patient's body that are of interest and which may be useful in evaluating gastrointestinal motility and/or transit time. For example, a single sensor may be strategically located on the patient's body and/or may be moved sequentially to different key locations on the patient's body to detect gastrointestinal sounds.

As discussed in detail below, the analyzer 24 can include amplifiers, filters, transient protection and other circuitry that amplifies signals sent by the sensors 22a, 22b, 22c, that attenuates noise signals, and/or that reduces the effects of aliasing. In particular, the analyzer 24 can include a low-pass filter having a cutoff frequency in the range of approximately 1100-1400 Hz. In one embodiment of the invention, the low-pass filter has a cutoff frequency in the range of approximately 1200-1300 Hz.

Alternatively or additionally, a high-pass filter can be incorporated within the analyzer 24. This high-pass filter may, for example, have a cutoff frequency in the range of approximately 70-90 Hz so that undesirable noise and sounds, such as muscle noise, breathing sounds, cardiac sounds, non-gastric gastrointestinal sounds or any other undesirable sounds or noise are substantially attenuated or eliminated before the signals sent by the sensors 22a, 22b, 22c are processed further.

The spectral energy of the most potentially corrupting non-gastrointestinal sounds is often in a frequency band of approximately 20-250 Hz. However, the amplitude of these corrupting sounds can be reduced, in some cases significantly reduced, for adult patients by considered positioning of the sensors 22a, 22b, 22c.

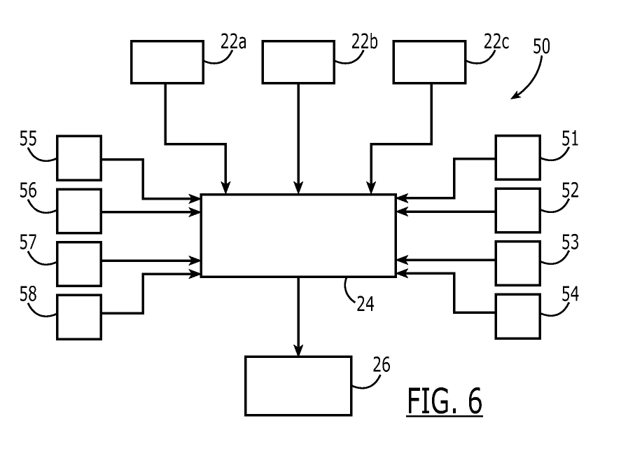

Referring now to FIG. 3, there is shown a preferred placement of sensors 22a, 22b, 22c, according to one embodiment of the invention. As illustrated in FIG. 3, sensor 22a is preferably placed in the upper left quadrant proximate the gastric fundus, sensor 22b is preferably placed in the lower right quadrant proximate the cecum, and sensor 22c is preferably placed in the lower left quadrant proximate the small intestine, more preferably, proximate the descending colon.

According to embodiments of the invention, the sensors 22a, 22b, 22c can be disposed in locations other than those specifically depicted in FIG. 3 without departing from the scope and the spirit of the invention. For example, sensor 22a can be located on a traverse line approximately two-thirds of the distance between the umbilicus and xyphoid to the right of the midline, sensor 22b can be located over the left coastal margin and sensor 22c can be located at the midline at approximately one-half of the distance between the umbilicus and symphosis pubis.

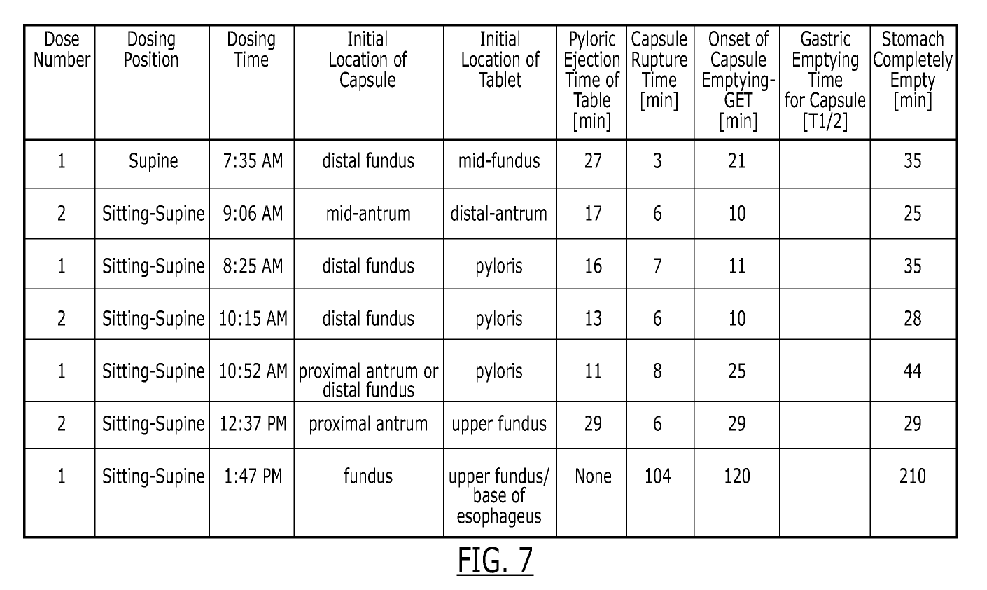

Referring to FIG. 4, according to one embodiment of the invention, the analyzer 24 is adapted to perform the following functions: (i) receive recorded acoustic energy (or gastrointestinal sound) signals from the sensors (e.g., sensors 22a, 22b, 22c) 30, (ii) store the signals in a memory medium 32, and (iii) process the acoustic energy signals 33 to, according to embodiments of the invention, derive at least one gastrointestinal parameter or gastrointestinal event (and/or occurrence thereof) relating thereto. In some embodiments of the invention, the analyzer 24 is further adapted to compare the gastrointestinal parameter or event to at least one physiological parameter, such as a pharmacokinetic (PK) parameter, that is induced in a subject by the administration of a pharmaceutical composition.

As illustrated in FIG. 4, the analyzer 24 is also adapted to provide at least one output signal 39 representing recorded acoustic energy and/or, according to further envisioned embodiments of the invention (discussed below), a physiological characteristic.

According to embodiments of the invention, the signal processing 33 includes the steps of: (i) filtering extraneous artifacts from the signals 34, (ii) determining a signal amplitude envelope based on the signals 36, and (iii) determining the dominant frequency of the signals 38.

According to the invention, the filtering step 34 can be performed with software, e.g., computer program, or hardware. Thus, in some embodiments of the invention, the analyzer is programmed to filter the acoustic energy signals and extract the frequency band of interest from the signals.

In one embodiment, the frequency of interest is in the range of approximately 70-1400 Hz. In another embodiment, the frequency of interest is in the range of approximately 90-1200 Hz.

According to the invention, various conventional programs can be employed within the scope of the invention to perform the noted filtering step 34.

In other embodiments of the invention, the filtering step 34 is performed via hardware. In one embodiment, the analyzer circuit includes high and low pass filters that are adapted to filter the extraneous artifacts from the signals 34. According to the invention, various high and low pass filters can be employed within the scope of the invention. In one embodiment, the high pass filter comprises a Blackman windowed, balanced 401-tap FIR with a cutoff set at 80 Hz and the low pass filter comprises a Blackman windowed, balanced 400-tap FIR with a cutoff set at 1250 Hz.

In one embodiment, the signal amplitude envelope is determined using a sliding Hilbert transform with a 5 μsec window. As is will known in the art, Hilbert transforms are commonly used to determine a signal envelope. See, e.g., T. Tomomasa, et al., “Gastrointestinal Sounds and Migrating Motor Complex In Fasted Humans”, The American Journal of Gastroenterology, Vol. 94, No. 2, pp. 374-381 (1999); J. Farrar, et al., “Gastrointestinal Motility as Revealed by Study of Abdominal Sounds”, Gastroenterology, Vol. 29, No. 5, pp. 789-800 (1955); which are incorporated by reference herein.

Applicants have found that the Hilbert transform smoothed out the short “pops”, i.e. intermittent acoustic energy spikes, and transformed the bipolar acoustic energy signals into a signal that can be readily analyzed using a simple V_{threshold}, as defined above.

According to embodiments of the invention, the dominant frequency of the acoustic energy signals can similarly be determined by various conventional means. In one embodiment, the dominant frequency was determined by isolating peaks >V_{threshold} for time >5 μsec.

Referring back to FIG. 2, according to the invention, the display means 26 can comprise any suitable medium that is capable of providing at least one visual display reflecting recorded acoustic energy signals (pre-and post-processed) and/or recorded physiological characteristics. In one embodiment, the display means 26 comprises a computer monitor.

According to other embodiments of the invention, the display means 26 can also comprise an audible display. The audible display can be adapted to provide a sound or tone representing, for example, a gastrointestinal event or a MMC phase. The audible display can be further adapted to provide different sounds or tones representing a selective gastrointestinal event or MMC phases or characteristic relating thereto, e.g., initiation of a phase.

The display means 26 can also provide at least one visual display representing recorded acoustic energy signals (pre-and post-processed) and/or recorded physiological characteristics, and at least one audible sound or tone representing at least one gastrointestinal event.

As will be appreciated by one having ordinary skill in the art, the display means 26 can also be an integral component or feature of the analyzer 24.

As will be appreciated by one having ordinary skill in the art, the sensors 22a, 22b, 22c of the invention can be positioned on a subject's body in various conventional means. By way of example, the sensors 22a, 22b, 22c can include an adhesive ring or surface on the housing that is adapted to temporarily engage the skin of the subject. The sensors 22a, 22b, 22c can also be attached to the subject's skin via a strip of medical tape or elastic bandage.

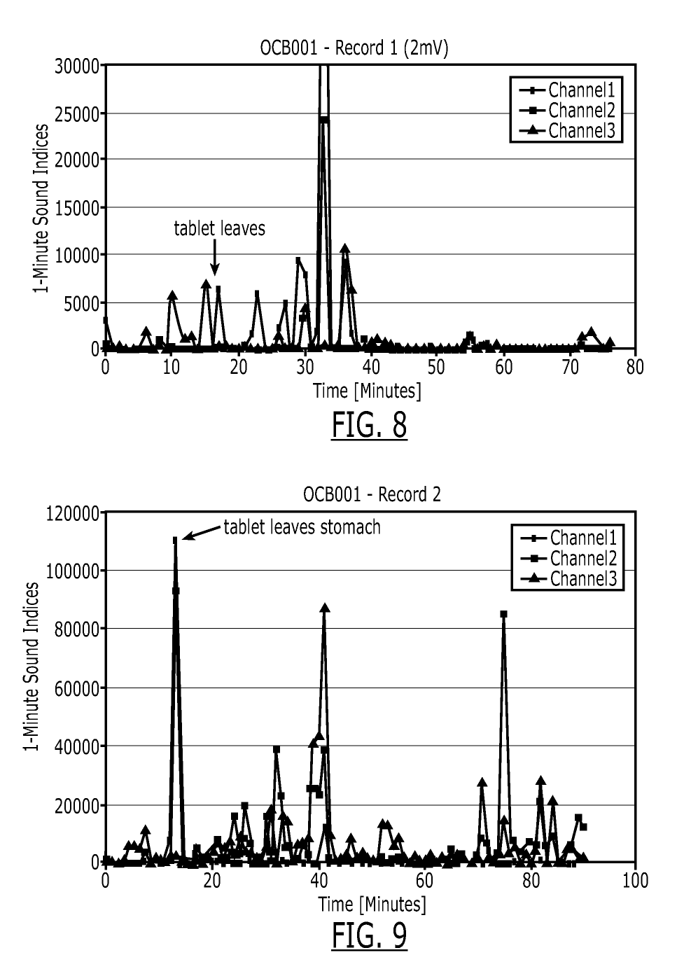

Referring now to FIG. 5, in one embodiment of the invention, the sensors 22a, 22b, 22c are positioned and maintained in a substantially static position against the subject's body via a vest 40. According to the invention, the vest 40 can comprise various sizes and materials.

In one embodiment, the vest 40 is adjustable and comprises a light weight, mesh material, e.g., nylon or lycra. In one embodiment of the invention, the vest 40 includes at least one pocket that is configured to receive and seat at least one sensor. Preferably, the vest 40 includes a plurality of pockets that are configured to receive and position a plurality of sensors; the pockets being positioned to correspond to selective positions on a subject's body when worn by the subject.

In another envisioned embodiment, the vest 40 and sensor(s) include a simple male-female snap system. In one embodiment, the vest 40 can include a plurality of positioned female portions of the snap system and the sensors can include a male portion that can engage and, hence, be secured on the vest 40 by the receiving vest female portions. In other embodiments, the vest 40 can include a plurality of positioned male portions of the snap system and the sensors can include a female portion that can engage and, hence, be secured on the vest 40 by the receiving vest male portions.

In the embodiment shown in FIG. 5, the vest 40 includes at least three pockets 42 adapted to receive and seat acoustic energy sensors 22a, 22b, 22c. The vest 40 also preferably includes an analyzer pocket 44 that is adapted to receive the analyzer 24.

As will be readily apparent to one having ordinary skill in the art, the vest 40 provides the system 20 with mobility.

In further envisioned embodiments of the invention, the gastrointestinal motility analysis system 20 includes at least one, preferably, a plurality of additional sensors that are adapted to record one or more physiological characteristics. Such physiological characteristics include, without limitation, ECG, pulse rate, SO₂, skin temperature, core temperature and respiration. A sensor (e.g., 3-axis accelerometer) can also be employed to monitor body position and/or movement.

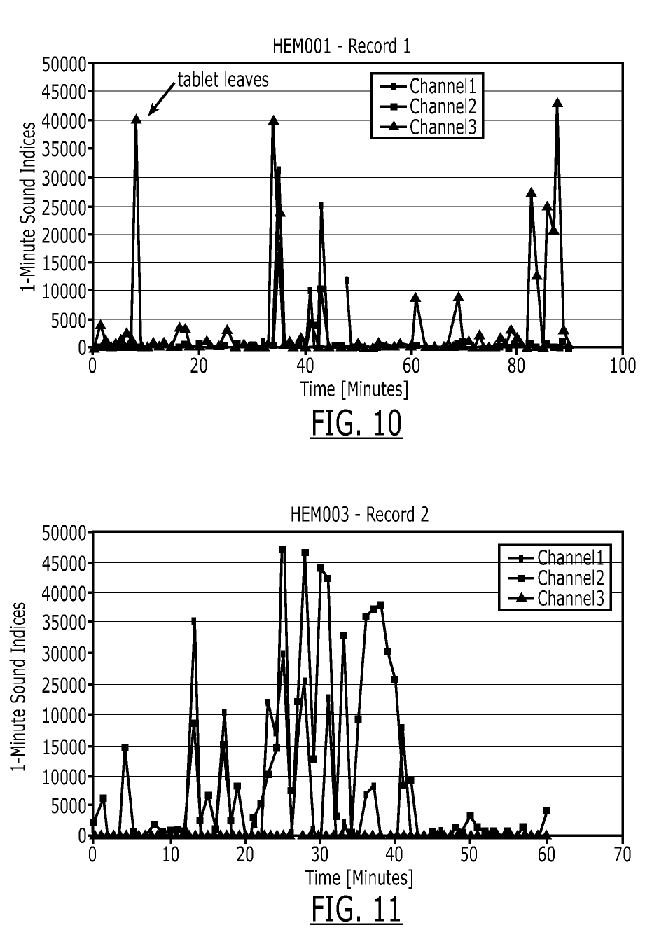

Referring now to FIG. 6, there is shown a schematic illustration of one embodiment of a gastrointestinal motility analysis system 50, according to the present invention, employing multiple function sensors 22a-22c and 51-58. In one embodiment of the invention, sensor 51 comprises an ECG sensor adapted to monitor cardiac performance and/or function, sensor 52 comprises a pulse rate sensor adapted to monitor the subject's pulse rate, sensor 53 comprises an SO₂ sensor adapted to monitor the subject's blood oxygen level, sensor 54 comprises a first temperature sensor adapted to monitor the subject's skin temperature, sensor 55 comprises a second temperature sensor adapted to monitor the subject's core temperature, sensor 56 comprises a respiration sensor that is adapted to monitor the subject's respiration rate and tidal volume, and sensor 57 comprises a position/motion sensor that is adapted to monitor the subject's movement and/or position.

As illustrated in FIG. 6, the system 50 also includes one additional sensor 58. In one embodiment, sensor 58 comprises an acoustic sensor that is adapted to monitor non-gastrointestinal related acoustic energy, such as a cough. According to the invention, the signals from the acoustic sensor can be used to identify and extract non-gastrointestinal related signals or artifacts that may have been recorded by the acoustic energy sensors 22a, 22b, 22c.

According to the invention, the additional sensors 51-58 can similarly be attached directly to the skin of the subject. The sensors 51-58 can also be incorporated into vest 40.

As indicated above, while the system 50 is shown with three acoustic energy sensors 22a, 22b, 22c, the system 50 can include less than three sensors, e.g., sensor 22a, or more such sensors.

It is also to be understood that while the system 50 is shown with eleven (11) sensors, i.e. sensors 22a-22c and 51-58, the system 50 can include any number of the sensors, e.g. one sensor, three sensor, six sensors, etc., and/or any combination of at least one of the sensors 22a-22c and zero or more of the sensors 51-58. For example, the system 50 can include sensors 22a, 22b, 52 and 57 or sensors 22a, 52 and 56.

As will be readily apparent to one skilled in the art, embodiments of the present invention can provide one or more advantages, such as:

The provision of a method and system for monitoring gastrointestinal motility that can be effectively employed during research of a pharmaceutical composition, and clinical trials related thereto, to better assess research and clinical data.

The provision of a method and system for monitoring gastrointestinal motility that has the potential to reduce the time and resources associated with research of a pharmaceutical composition and clinical trials related thereto.

The provision of a method and system for monitoring gastrointestinal motility that can be readily employed by a medical practitioner as a diagnostic aid during assessments of gastrointestinal behavior.

### EXAMPLES

The following examples are provided to enable those skilled in the art to more clearly understand and practice the present invention. They should not be considered as limiting the scope of the invention, but merely as being illustrated as representative thereof.

### Example 1

Six male subjects were initially provided with a health assessment. All subjects passed the initial health assessment. The subjects then spent the night at the test center and fasted (i.e. water only) for at least 11 hours before the study began.

Three acoustic energy sensors were positioned on each subject. Sensor #1 was placed 4-6 inches below the patient's right nipple, i.e. proximate the gastric fundus. Sensor #2 was placed 11-11.5 inches below the right nipple, i.e. proximate the cecum. Sensor #3 was placed in the Lower Left Quadrant, approximately 11 inches below the left nipple, i.e. proximate the loudest part of the small intestine/descending colon. The sensors were held firmly against each subject's body by a lightweight, close fitting nylon mesh vest, such as vest 40.

The sensors were custom modified Welch-Allen Master Elite Plus Stethoscopes. Unlike traditional stethoscopes, these pressure-based microphones employ a technology that is less sensitive to indirect vibration and hence ambient noise. Further, the sensors also contain signal processing circuitry that improves signal-to-noise ratio and deliver either traditional audio, or mono line-out signals.

Without disturbing the microphone head or signal processing electronics, the housing was removed and the microphones were repackaged passing the power and line-out signals to custom front-end analog electronics with long wiring that allows for patient mobility. Additionally, the volume was set at maximum and the onboard filtering was set to “all-pass”, which encompasses a frequency band in the range of 100-1200 Hz.

All microphone channels were amplified and low-pass filtered via an analog 2-pole 1200 Hz low-pass Bessel filter, and then sampled onto a National Instruments DAQPad-6015 at 8000 Hz. Data was recorded in 10-minute segments and post processed via software written in National Instruments LabVIEW 7.1.

Gamma scintigraphy was also performed simultaneously to assess gastrointestinal motility. A dissolvable hard gelatin capsule and a non-disintegrating tablet with radioactive markers (¹¹¹InCl₃ and ^{99m}Tc-DTPA, respectively) were administered to each subject. The tablet and the capsule were taken simultaneously with a glass of water, since it is known that capsules taken without water can stick to the esophagus for up to two hours.

As is well known in the art, the radionucleotide markers emit gamma rays of different characteristic energies. Thus, the tablet and capsule's contents could be separately tracked.

Removable stickers containing small point sources of ¹¹¹InCl₃ encased in plastic were placed on the chest and hip of each subject as a reference to ensure consistent placement of the subject under the gamma camera in between pictures. Pictures were taken every 20 seconds and integrated images stored every 1 minute by the gamma scintigraphy system. Tablet and capsule position in the gastrointestinal tract were determined and recorded for subsequent analysis.

Gastrointestinal sounds were also recorded during the ingestion of the dissolvable hard gelatin capsule and a non-disintegrating tablet. The recorded sounds i.e. sound files were stored in an analyzer according to embodiments of the invention. The sound files were processed as discussed above.

During the first of the study, the subjects were asked to remain quiet in a supine position under the gamma scintigraphy camera. Several parameters were subsequently analyzed. Individual sound dominant frequency, duration, and intensity were also all calculated.

The Sound Index (or SI) was also calculated. SI, as used herein, means the sum of the absolute amplitudes for all detected sound over a one (1) minute time period, expressed as mV/min.

As discussed above, it is known that in a fasted state, upper tract digestion occurs in a 4-stage cyclic pattern with the largest contractions of the stomach (i.e., Phase 3) usually initiated with a migrating motor complex (MMC) that proceeds from the stomach towards the ileum of the small intestine. The period between MMC's has been well established and is typically around 2 hours (although times ranging from 1 to 3 hours are not uncommon).

During the studies, clearly identifiable MMC's were observed in most subjects (˜66.7%) as identified by large SI's in all three sensors; with Sensors #1 and #2 being the loudest.

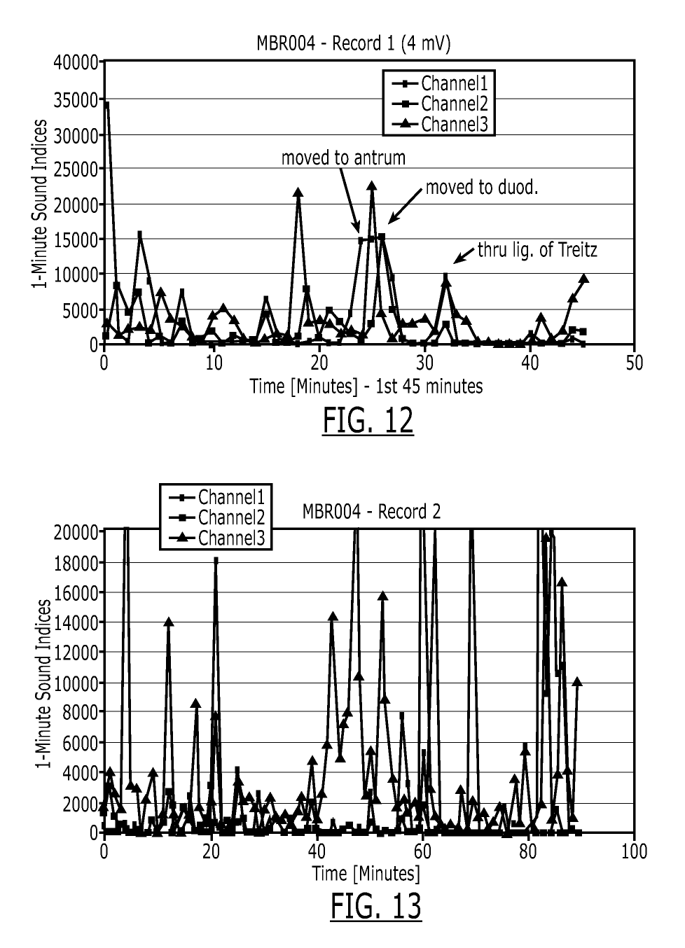

Referring now to FIG. 7, there is shown a summary of the gamma scintigraphy assessment. As reflected in FIG. 7, in all studies, but one, i.e. an “outlier”, gamma scintigraphy determined that the tablets were ejected from the stomach between 11-29 minutes (mean 18.88 min). Thus, it can be inferred that the test tablets were passed with the liquid from the stomach.

Interestingly, the “outlier” displayed an MMC without tablet movement. Tablet movement within the stomach only came later corresponding to a large sound and SI in channel 1 around 1 hour, 40 minutes. Complete tablet ejection did not occur during the entire duration of the study, i.e. 5 hours and 51 minutes. The cause of this is uncertain, but highlights the need for gastrointestinal transit monitoring.

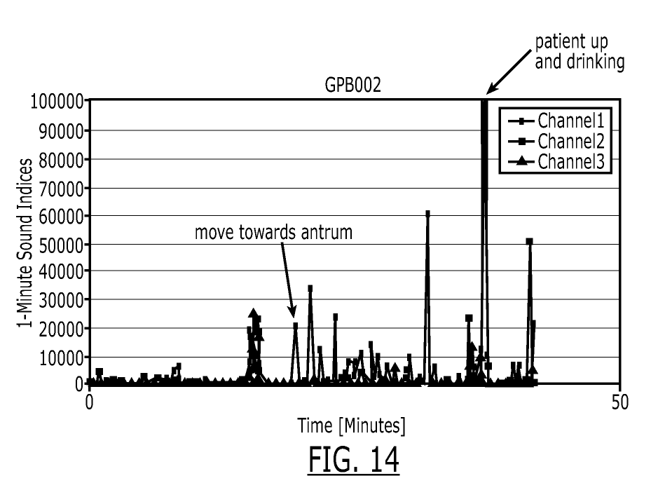

Referring now to FIGS. 8-14, there are shown graphs reflecting the sounds recorded by the sensors, i.e. minute sound indices versus time. As reflected in FIGS. 8-14, in all 6 studies, where gastric emptying of the tablet did occur during monitoring, significant bowel sounds and SI's were recorded at the time of emptying. In 5 subjects, channel #1 (or Sensor #1), which monitored gastric sounds, produced the highest SI recorded to that point.

Tablets that landed in the antrum, where muscular activity occurs, moved corresponding to first large sound in channel #1. Tablets that landed in the antrum generally took two or three large SI's to move, with the first or second SI corresponding to movement into the fundus.

For the “outlier”, bowel sounds generally matched the scintigraphy data. There appeared to be an MMC around 1 hour, 40 minutes (i.e. strong signal in all three channels), which did not affect the tablet's movement. However, afterwards, the next considerable SI was incident with movement of the tablet from the initial location of upper fundus to the antrum of the stomach. Afterwards, there were intermittent sounds recorded in channel 1, but no tablet movement. Notable though, are the very low levels of sound in the other two sensors, implying overall gastrointestinal quiescence.

The results of this study reflect that in subjects that are quiet in a supine position, discernable bowel sounds recorded by a sensor of the invention correspond to tablet ejection, as shown by gamma scintigraphy. Indeed, movements of tablet position (antrum or fundus) in the stomach were also marked by large sounds. Overall quiet sounds were detected in the patient who never experienced gastric tablet ejection. MMC's were also clearly identifiable in several subjects.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A system for monitoring gastrointestinal motility of a subject, comprising:
at least one sensor mountable proximate a body region of the subject, said sensor being adapted to sense acoustic energy and generate at least one acoustic energy signal representing said acoustic energy; and
a processing unit adapted to receive said acoustic energy signal, said processing unit being further adapted to process said acoustic energy signal and determine the occurrence of at least one gastrointestinal event therefrom.

2. The system of claim 1, wherein said gastrointestinal event is selected from an event consisting of gastrointestinal mixing, emptying, contraction and propulsion.

3. The system of claim 1, wherein said sensor generates a plurality of acoustic energy signals representing said acoustic energy.

4. The system of claim 3, wherein said processing unit is adapted to receive and process said acoustic energy signals and determine the occurrence of at least one gastrointestinal event therefrom.

5. The system of claim 1, wherein said acoustic energy comprises gastrointestinal sounds.

6. A method of monitoring gastrointestinal motility of a subject, comprising the steps of:
sensing acoustic energy generated by the subject's gastrointestinal system, and
processing the acoustic energy to derive a gastrointestinal parameter.

7. The method of claim 6, wherein said gastrointestinal parameter comprises a gastrointestinal event.

8. The method of claim 7, wherein said gastrointestinal event is selected from an event consisting of gastrointestinal mixing, emptying, contraction and propulsion.

9. The method of claim 6, wherein said gastrointestinal parameter comprises gastrointestinal transit time.

10. A method for evaluating clinical data derived from a subject, comprising the step of comparing a gastrointestinal parameter of the subject to at least one physiological parameter induced in the subject by the administration of the pharmaceutical to the subject.

11. A method for evaluating clinical data derived from a subject, comprising the steps of:
orally administering a pharmaceutical to the subject, said pharmaceutical including a pharmaceutical composition;
monitoring acoustic energy generated by the subject's gastrointestinal system;
generating at least one acoustic energy signal representing said acoustic energy;
processing said acoustic energy signal to derive a gastrointestinal parameter related thereto; and
comparing said gastrointestinal parameter to at least one physiological parameter induced in the subject by the administration of said pharmaceutical to the subject.

12. The method of claim 10, wherein said gastrointestinal parameter comprises gastrointestinal transit time.

13. The method of claim 10, wherein said physiological parameter comprises a pharmacokinetic (PK) characteristic.

14. A method for evaluating gastrointestinal motility of a subject, comprising the steps of:
orally administering an ingestible to the subject;
monitoring acoustic energy generated by the subject's gastrointestinal system;
generating at least one acoustic energy signal representing said acoustic energy; and
processing said acoustic energy signal to derive a gastrointestinal parameter related thereto.

15. The method of claim 13, wherein said gastrointestinal parameter comprises gastrointestinal transit time.
